(19) United States
(12) Patent Application Publication
(10) Pub. No.: US 2024/0284845 A1
(43) Pub. Date: Aug. 29, 2024

(54) NATURAL FIBER-BASED PROPAGATION MEDIUM

(71) Applicant: PROFILE PRODUCTS LLC, Buffalo Grove, IL (US)

(72) Inventors: Michael Dan ROBESON, Windsor, CO (US); Daniel Scott NORDEN, Belton, SC (US); Stanton Reid SMITH, Maiden, NC (US); Gary Lane BOWERS, Jonesborough, TN (US); Ryan Michael Knauer, Hickory, NC (US); Timothy Daniel Knauer, Charlotte, NC (US)

(73) Assignee: PROFILE PRODUCTS LLC, Buffalo Grove, IL (US)

(21) Appl. No.: 18/587,413

(22) Filed: Feb. 26, 2024

Related U.S. Application Data
(60) Provisional application No. 63/486,867, filed on Feb. 24, 2023.

(51) Int. Cl. A01H 4/00

(52) U.S. Cl. CPC A01H 4/002

(57) ABSTRACT

A fibrous propagation medium includes a volume of refined, acidic wood and/or bark fiber having about 2.3-5.0 vol. % of a particle size of 2.36-4.74 mm (sieve #8), based on the total volume of the wood fiber, moisture content of about 16-28 wt. %, and water holding capacity of about 50-95%, according to the NCSU Promoter Analysis.

### CROSS-REFERENCE TO RELATED APPLICATION

This application claims the benefit of U.S. Provisional Application Ser. No. 63/486,867 filed on Feb. 24, 2023, the disclosure of which is hereby incorporated by reference herein in its entirety.

### TECHNICAL FIELD

The present disclosure relates to a natural fiber-based propagation medium and methods of making and using the same.

### BACKGROUND

Plant propagation relates to a process by which new plants grow from a variety of sources such as seeds, cuttings, and other plant parts. To support the initial germination and growth, various media have been developed.

### SUMMARY

In one embodiment, a propagation fiber medium is disclosed. The medium may include wood fiber and bark fiber in a ratio of about 80-90 to 10-20 wt. %, based on the total weight of the medium. About 0.23-0.50 vol. % of the wood fiber and bark fiber may have a particle size of 2.36-4.74 mm (sieve #8). The medium may be a malleable medium insertable into a propagation container.

In an alternative embodiment, a fibrous propagation medium is disclosed. The medium may include a volume of refined, acidic wood and/or bark fiber having about 2.3-5.0 vol. % of a particle size of 2.36-4.74 mm (sieve #8), based on the total volume of the wood fiber. The medium may have moisture content of about 16-28 wt. %. The medium may have water holding capacity of about 50-95%, according to the NCSU Promoter Analysis. The medium may also include about 25-31.1 vol. % of a particle size of 1.18-2.35 mm (sieve #16), based on the total volume of the wood fiber. The wood and/or bark fiber may include pine wood fiber. The medium may have loose bulk density of about 1.3-3.0 lbs/ft³ (20.8-48.1 kg/m³). The medium may have structural integrity when inserted into a cavity formed by a wall. The medium may be configurable into a self-supporting 3-dimensional shape without any added binder. The medium may be free of an added binder, surfactant, or both.

In yet another embodiment, a propagation medium is disclosed. The medium may include a blend of wood and/or bark fiber having about 2.3-5.0 vol. % of a particle size of 2.36-4.74 mm (sieve #8), based on the total volume of the wood fiber and peat having about 3.1-7.0 vol. % of a particle size of 2.36-4.74 mm (sieve #8), based on the total volume of the peat. The propagation medium may have moisture content of about 40-70 wt. % and water holding capacity of about 50-95%, according to the NCSU Promoter Analysis. The medium may include less than about 5 wt. or vol. % additional materials including one or more of heat-treated mineral particles, pigments, pigment precursors, fertilizer(s), macronutrient(s), micronutrient(s), mineral(s), mineral particle(s), biostimulant(s), interlocking manmade fiber(s), interlocking biodegradable fiber(s), surfactant(s), or seed(s), based on the total weight or volume of the propagation medium. The wood and/or bark fiber:peat vol. % ratio may be about 50:50 to 90:10. The wood and/or bark fiber may include pine wood fiber. A ratio of water holding capacity to porosity of the blend may be about 50:99% to 98:85%. The propagation medium may also include a fiber including one or more of coir, sisal, jute, straw, wheat straw, rice hulls, composted bark, alfalfa, flax, hammermilled fiber such as hammermilled tree substrate, hammermilled pine tree substrate, sawdust, compost, manure, paper, recycled paper, or cellulose fibers. The medium may have volume of air space of about 10-40%, according to the NCSU Poromoter Analysis.

In an alternative embodiment, a propagation mix is disclosed. The propagation medium may include a homogenous blend of a first volume of refined, added-binder-free wood and/or bark fiber having about 2.3-5.0 vol. % of a particle size of 2.36-4.74 mm (sieve #8), based on the total volume of the wood and/or bark fiber, moisture content of about 16-28 wt. % and a second volume of one or more of heat-treated mineral particles, pigments, pigment precursors, fertilizer(s), macronutrient(s), micronutrient(s), mineral(s), mineral particle(s), biostimulant(s), interlocking manmade fiber(s), interlocking biodegradable fiber(s), surfactant(s), a second type of fiber. The second volume may include the second type of fiber including one or more of peat, coir, sisal, jute, straw, wheat straw, rice hulls, composted bark, alfalfa, flax, hammermilled fiber such as hammermilled tree substrate, hammermilled pine tree substrate, sawdust, compost, manure, paper, recycled paper, cellulose fibers. The homogenous blend may include about 50-65 vol. % peat and 35-50 vol. % wood and/or bark fiber. The mix may include seeds. The propagation mix may have volume of air space of about 10-40%, according to the NCSU Porometer Analysis. The first volume:second volume ratio may be about 50:50 vol. % to 90:10 vol. %.

### BRIEF DESCRIPTION OF THE DRAWINGS

FIG. 1 is a photograph of a non-limiting example of a propagation fiber according to one or more embodiments, the example including a mixture of wood fiber and bark fiber having a particle size distribution described herein;

FIG. 2 shows a microscale photograph of individual fibers of a non-limiting example wood/bark fiber propagation medium according to one or more embodiments disclosed herein;

FIG. 3 shows a comparative microscale photograph, at the same magnification as FIG. 2, of a non-propagation medium wood/bark fiber having a different particle distribution than the fiber shown in FIG. 2;

FIG. 4 is a photograph of a non-limiting example propagation fiber disclosed herein in a bale before expansion and insertion into propagation trays;

FIG. 5 shows a non-limiting example of loose form 100 wt. or vol. % wood/bark fiber propagation medium disclosed herein;

FIG. 6 shows a non-limiting example of loose form blend of peat:wood/bark fiber in the ratio of 65:35 vol. %;

FIG. 7 is a photograph of strawberry plants grown in a medium of Example 23 (on the right) and in a control (on the left);

FIG. 8 is a photograph of begonias propagated in the medium of Example 24 after removal from trays;

FIGS. 9A and 9B are photographs of begonias propagated in a control, demonstrating lack of structural integrity after removal from trays;

FIGS. 10A and 10B, are photographs of non-limiting example trays with cells including the propagation medium disclosed herein;

FIGS. 11A-D are photographs of the propagation medium disclosed herein of Example 25 after removal from trays;

FIGS. 12A and 12B are photographs of the propagation medium disclosed herein of Example 26, each medium including a plug holder;

FIGS. 13A and 13B are photographs of the propagation medium disclosed herein of Example 27 (on the right) and control (on the left) after removal from trays; and

FIGS. 14A and 14B are photographs of the propagation medium disclosed herein of Example 28 (on the right) and control (on the left) demonstrating erosion of the control after removal from the tray.

### DETAILED DESCRIPTION

Embodiments of the present disclosure are described herein. It is to be understood, however, that the disclosed embodiments are merely examples and other embodiments may take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present embodiments. As those of ordinary skill in the art will understand, various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except in the examples, or where otherwise expressly indicated, all numerical quantities in this description indicating amounts of material or conditions of reaction and/or use are to be understood as modified by the word “about” in describing the broadest scope of the disclosure. Practice within the numerical limits stated is generally preferred. Also, unless expressly stated to the contrary: percent, “parts of,” and ratio values are by weight; the description of a group or class of materials as suitable or preferred for a given purpose in connection with the disclosure implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “substantially,” “generally,” or “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the disclosure can be obtained within a range of +/−5% of the indicated value. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4, . . . , 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. Similarly, whenever listing integers are provided herein, it should also be appreciated that the listing of integers explicitly includes ranges of any two integers within the listing.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

As used herein, the term “and/or” means that either all or only one of the elements of said group may be present. For example, “A and/or B” means “only A, or only B, or both A and B”. In the case of “only A,” the term also covers the possibility that B is absent, i.e. “only A, but not B”.

It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

The term “comprising” is synonymous with “including,” “having,” “containing,” or “characterized by.” These terms are inclusive and open-ended and do not exclude additional, unrecited elements or method steps. The term “including” or “includes” may encompass the phrases “comprise,” “consist of,” or “essentially consist of.”

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed subject matter can include the use of either of the other two terms.

The term “one or more” means “at least one” and the term “at least one” means “one or more.” The terms “one or more” and “at least one” include “plurality” as a subset.

The description of a group or class of materials as suitable for a given purpose in connection with one or more embodiments implies that mixtures of any two or more of the members of the group or class are suitable. Also, the description of a group or class of materials as suitable for a given purpose in connection with one or more embodiments implies that the group or class of materials can “comprise,” “consist of,” and/or “consist essentially of” any member or the entirety of that group or class of materials. First definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

In agriculture and horticulture, plant propagation relates to a process by which new plants grow from a variety of sources, specifically seeds, cutting, and other plant parts. Propagation is part of a plant growing cycle and includes different steps and stages. The stages may be summarized as seed to sprout, a seedling, vegetative stage, budding, flowering, and ripening.

As each plant has changing needs during its growth and development, a generic medium used for all stages is not the most optimal or efficient. While a medium may be fit for a single stage, extending its use as a sole medium for multiple stages may result in slower growth, lower yield, or other undesirable conditions in comparison to moving the plant through different media based on the plant's stage. An additional consideration is the container size. Starting the plants in smaller container sizes allows the grower to maximize space. The smaller cells in trays at high density are often used to maximize greenhouse space efficiency. Smaller containers/cells typically require different media to fill than larger containers.

Therefore, in a commercial setting, plants in different stages are provided different environments and media. As the seed or cutting grows into a seedling and later a plant, it may be replanted or transferred into a medium which offers increased space for growth, additional nutrition, different physical properties, or a combination thereof. More than one transplantation may be required for the same plant, catering to the changing needs of the developing and maturing plant.

Depending on the grown type or species of plant, different types of containers may be implemented for the initial stage including flats, trays, containers, pots, or the like. Traditionally, seeds may be started in container or germination trays. The trays are containers with a number of compartments or cells, typically of the same size, which may hold a medium and support a growing seedling/plant. The medium may be relatively loose prior to insertion into the cells. When empty, the trays are typically stackable. The trays are commonly made of plastic, but may be made from other materials such as composite, silicone, rubber, bamboo, wood, cardboard, etc. As the plant develops, the root system of the plant develops in the medium within the confines of each cell. The developing plant is then transplanted into a tray with larger cells, into pots, grow cubes or slabs, or other types of media structured to sustain additional growth. Alternatively, the plant may be transplanted outdoors into soil. At the point of transplant, the root system typically envelops the medium and binds it together such that the medium with the roots form a relatively compact structure.

An alternative way to start plants from seeds or cutting is using a grow plug. A grow plug is a self-supporting structure made of a medium in which a plant can be germinated and/or grown into a seedling. The grow plug medium differs from the tray medium in at least one aspect. The trays, mentioned above, provide physical structure to a medium which is inserted within the cells of the trays in which the plant is propagated. The medium in the trays typically does not provide physical support to the growing plant. In contrast, the grow plug provides physical support by itself and thus does not need to be inserted within another physical structure. Yet, the plugs may be insertable within the trays and/or may be used in the cells of the trays. Because the grow plug medium provides physical structure by itself, the grow plug medium typically has a different composition than a relatively loose medium insertable into the trays.

Furthermore, the grow plugs are typically used to grow higher value crops than the medium used in the propagation trays. A non-liming example of a high value crop typically grown in a grow plug may be lettuce. The grow plugs may be also used to support plants which do not develop an extensive root system such as lettuce. Once such plant develops to a degree which renders it ready for transplant, the entire plug may be lifted and inserted within the next level container, pot, grow cube, etc. Due to the requirements of the self-supporting physical structure, the grow plug is typically more expensive than the relatively loose medium insertable within the propagation trays. The physical structure is typically achieved with an addition of a binder. Additionally, special equipment may be needed to manage the plugs which may add to the operational costs.

The first stage of plant growth and development thus needs a specifically tailored material. Different types of media have been used for the first stage propagation. The most typical example are peat mixtures. Peat by itself may harden and become too dense with time. Additionally, peat may hold undesirable amounts of water and lack sufficient air space. Therefore, it has been provided in a mixture with other materials such as soil, sand, vermiculite, perlite, or the like. Furthermore, traditional peat harvesting has undergone more recent scrutiny as a potential environmental concern with harvested, unrehabilitated peat bogs contributing to released carbon dioxide, otherwise stored in the undisturbed bogs. Thus, using lesser quantities of peat is desirable.

Additional disadvantages traditionally associated with the propagation materials include undesirably high levels of moisture content, fragility of the propagation materials throughout transplanting stages, and turbidity of water. Furthermore, some propagation media are difficult to extract from the trays when time comes for transplanting. Such media adhere to the sides of the cells or containers with too much force, which may result in compromised root system of the transplant and/or structurally compromised propagation medium. Some media also succumb to erosion from irrigation and do not have sufficient structural integrity to be ejected from the tray cell in one piece.

With the growing population of the world requiring food for its sustenance, there is a demand for efficient, accelerated ways to grow fruits and vegetables. Thus, there is a growing need in the agricultural and horticultural industries to provide a sustainable, renewable material which may be used as a propagation material for various plant species, and specifically a propagation material for the first stage(s) of plant development and growth.

In one or more embodiments, a propagation medium is disclosed. The propagation medium overcomes one or more drawbacks described above. The propagation medium may be used as a propagation material in trays, flats, cells, containers, troughs for plant propagation including seed or spore germination, striking, cuttings, or offsets propagation, microgreens propagation, root development, sprout or shoot development, seedling growth, tissue culture plant development, or the like. The propagation medium may be utilized to support various species such as leafy greens, cruciferous vegetables, marrow vegetables, root vegetables, edible plant stems, allium, berry plants, vines, ornamentals, annuals, perennials, or the like. The propagation medium, blend, or mix discussed herein may include seeds.

The propagation medium may include propagation material in a relatively loose form such that a volume of the propagation material may be insertable within a cell of a tray and be moldable to the shape of the cell.

The propagation medium may include one or more components. The propagation medium or material may include a first portion, volume, weight, or mass. The first portion, volume, weight, or mass may include fiber also called propagation fiber. The propagation fiber may be used by itself to form the propagation medium or material. The propagation fiber may include wood fiber, bark fiber, or both.

The propagation medium or material may also include a second portion, volume, weight, or mass. The second portion, volume, weight, or mass may include at least a second type of fiber, one or more non-fibrous components, or a combination thereof. The first and second portion, volume, weight, or mass may be combined to form a blend. The blend may be homogenous or heterogenous. The blend thus relates to a propagation medium having the wood/bark fiber of the first portion and at least one additional type of fiber, at least one non-fibrous component, or a combination thereof of the second portion. The term propagation medium/material encompasses a propagation fiber and a blend.

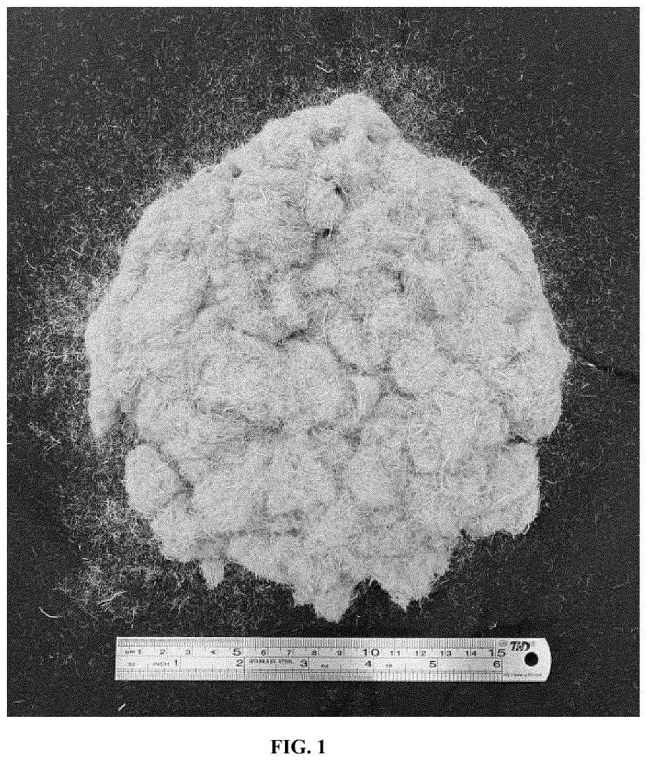

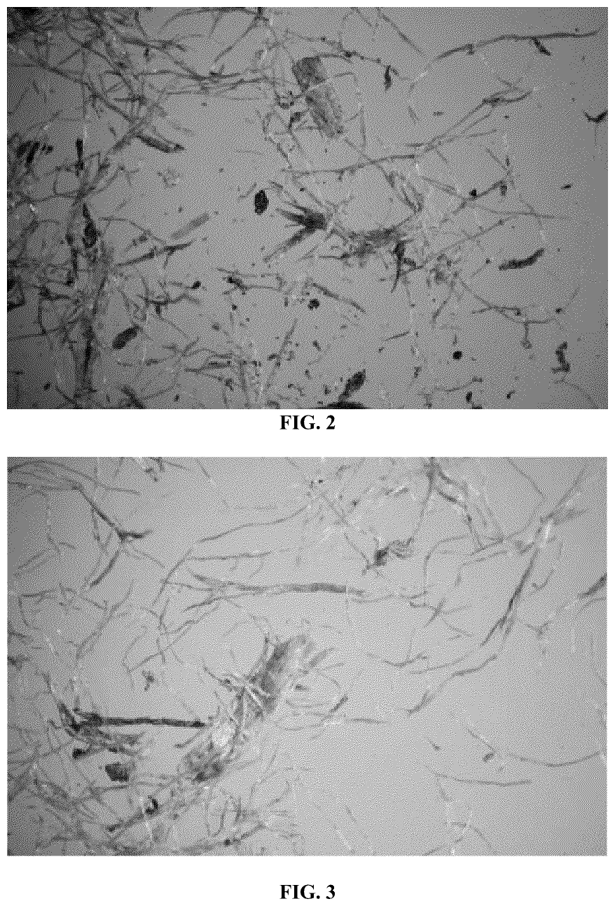

A non-limiting example of the fiber in a loose form is shown in FIG. 1. The non-limiting example of FIG. 1 shows a propagation fiber whose composition is wood fiber and bark fiber in a ratio of about 80-90:10-20% by volume, based on the total weight of the fiber. FIG. 2 provides a microscopic image of a portion of the non-limiting example propagation fiber. As can be observed in FIG. 2, the particle distribution includes a significant proportion of very small particles and a small proportion of relatively large, elongated particles. The small particles may assist in the herein-disclosed propagation fiber's ability to fill in containers and conform to various shapes of containers such as tray cells. Yet, presence of a predetermined amount of longer particles may enable binding of the fibers within the cells and between the fibers of the herein-disclosed propagation fiber. Hence, in at least one embodiment, while the propagation fiber lacks any added binders, or is free or any added binders, the fiber may possess binding properties unique to the herein-disclosed fiber. Moreover, it at least another embodiment, while the propagation fiber lacks any significant amount of added binders, or is substantially free or any added binders, i.e., less than 2 weight or volume % added binder, based on the total weight of the propagation fiber, the fiber may possess binding properties unique to the herein-disclosed fiber.

The binding capability of the herein-disclosed propagation fiber may be especially helpful for germinating seedlings or cuttings of plants with traditionally weaker root systems. The binding capabilities may thus assist such plants in root and plant stability during transplanting into the next stage media.

Without limiting this disclosure to a single theory, it is believed that the fibers of the propagation medium, once inserted into the tray cavities, physically communicate with one another, interlock, entangle, or otherwise form mechanical connections. The mechanical connections between fibers assist with the shape and structure maintenance. Additional advantages stem from the structural integrity of the growing medium configured or configurable into plugs or other 3-dimensional self-supporting shapes of the growing medium, for example maintenance of volume within which the root structure can develop and decreased, minimized, or diminished erosion of the medium when in contact with water.

In comparison to FIG. 2, FIG. 3 shows a post initial propagation fiber (or non-propagation fiber) including fibers with a much higher proportion of large particle fibers such as those in sieve #8. The term “post initial propagation” relates to stages of plant growth beyond the initial stage of seed or cutting start and initial growth. An example post initial propagation growing medium may be a grow cube or a grow slab designed to support growth after seed germination. Small particles shown in FIG. 2 are virtually absent in FIG. 3. The medium depicted in FIG. 3 may be well-suited to grow mature plants, but the absence of relatively small particles, and presence of relatively large, robust stringy fibers may diminish its suitability for propagation purposes.

In a cell, the herein-disclosed propagation fiber may be compressed, either alone, or as is typically the case after being blended with additional materials. Unlike the grow plug which retains shape by including one or more binders, the propagation fiber disclosed herein may be free, or essentially free, of any added binders, synthetic or natural. The absence of an added binder may provide several advantages such as biodegradability, environmental safety, ability to mix the medium with additional materials prior to insertion into trays and thus customization of the grow mix by a customer depending on the types of plants, conditions of the growth environment, resources availability, etc. Additionally, the loose form of the medium/material/fiber may be inserted into a container of any shape, and be molded to the bottom and sides of any container. The propagation medium/material disclosed herein thus features versatility which the traditional grow plugs do not typically offer.

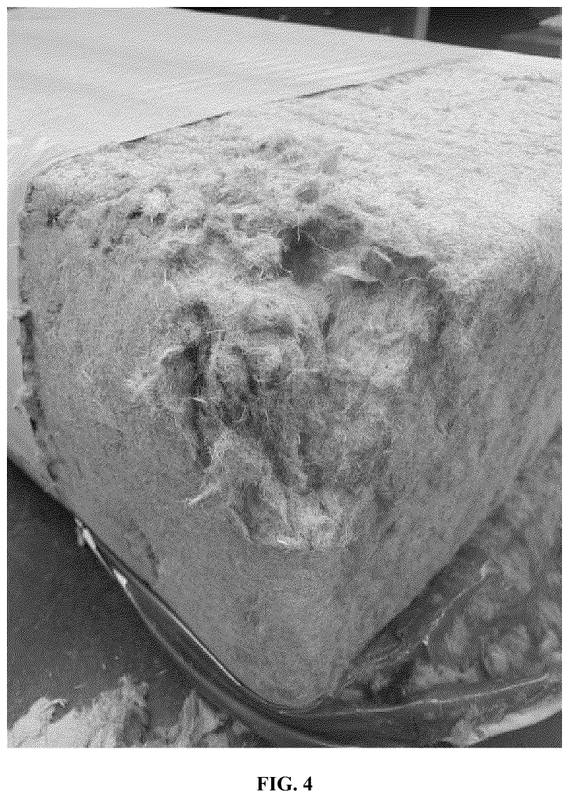

Additionally, the production process may produce a partially compressed fiber, which may be further expanded and compressed into the containers. The expansion may produce the loose form of the fiber described above. A non-limiting example of the partially compressed fiber in a bale is shown in FIG. 4. The packaged bale in FIG. 4 was opened to reveal the fiber within the packaging. The propagation fiber in FIG. 4 was photographed prior to expansion in a fiber opener. The bale of FIG. 4 shows fiber compressed for storage and transport, it does not represent a grow slab.

The disclosed propagation fiber may be made sterile, sterilized, or pasteurized such that the fiber may be free of unwanted pathogens. Likewise, the propagation medium/fiber may be free of unwanted metals, heavy metals, toxins, human pathogens, objects, residues of herbicides, pesticides, insecticides, etc. to comply with standards for food safety.

The propagation medium includes the first portion, volume, weight, or mass of wood and/or bark fiber. The wood fiber of the first portion, volume, weight, or mass of the medium may account for about 80-100, 86-98, or 88-96 wt. or vol. %, based on the total weight or volume of the first portion. The wood fiber of the first portion of the medium may account for about, at least about, or at most about 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, or 100 wt. or vol. %, based on the total weight or volume of the first portion. The propagation medium may include substantially only or 100 wt. or vol. % wood fiber, based on the weight or volume of the first portion.

The bark fiber of the first portion, volume, weight, or mass of the medium may account for about 0-20, 1-15, or 5-10 wt. or vol. %, based on the total weight or volume of the first portion. The bark may account for about, at least about, or at most about 0, 1, 2, 3, 4, 5, 6, 7, 8, 9, 10, 11, 12, 13, 14, 15, 16, 17, 18, 19, or 20 wt. or vol. %, based on the total weight or volume of the first portion. The first portion may be substantially free of bark or include about 0 wt. or vol. % of bark, based on the total weight or volume of the first portion.

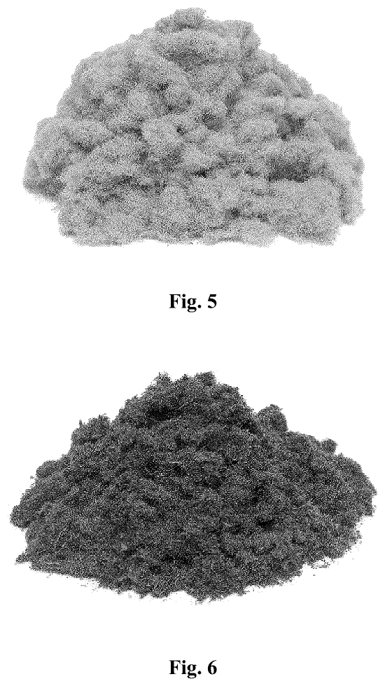

A non-limiting example of the first portion is shown in FIG. 5; the fiber includes 100 vol. % of wood fiber, based on the total weight of the medium. Another non-limiting example of the first portion is shown in FIG. 1; the fiber includes 80-90 vol. % wood fiber and 10-20 vol % bark.

The wood fiber may be made from various types of wood species such as softwood, hardwood, or both. Non-limiting example species may include pine such as Ponderosa, Sugar, White, and Yellow varieties of pine fiber, Hoop pine, Slash pine, Bunya pine, Kauri pine, Wollemi pine, Mediterranean pine, Monterey pine, Caribbean pine, Queensland pine, Norfolk Island Pine, Swiss pine, Turkish pine, Canary Island pine, Aleppo pine, Bosnian pine, Mountain pine, European black pine, Austrian pine, Macedonian pine, Maritime pine, Stone pine, Scots pine, poplar such as yellow poplar, spruce, cedar such as Western red cedar, fir such as Douglas fir, redwood such as California redwood, oak such as red oak, the like, or a combination thereof.

The bark fiber may be made from softwood, hardwood, or both. The term “bark fiber” refers to a fiberized plurality of stem tissues including one or more of cork (phellum), cork cambium (phellogen), phelloderm, cortex, phloem, vascular cambium, and xylem. Examples of useful tree barks include, but are not limited to, bark from trees mentioned above such as pine including species named above, spruce, oak, walnut, mahogany (Swietenia macrophylla, Swietenia mahagoni, Swietenia humilis), hemlock, Douglas fir, alder, elm, birch, Sitka spruce, sycamore, the like, or a combination thereof.

It is desirable that the consistency of the propagation medium is such that the fiber fits within the relatively confined spaces of propagation tray cells. The fiber is thus structured to have a particle size distribution allowing it to fit within the cells, to insert a certain volume of the fiber within the cells, to fill the cells, or a combination thereof. The fiber is also structured to provide adequate support for the root system which will develop once the medium is in use. The medium should have such density, volume, air capacity that together with a developed root system, the medium has structural integrity and can be transplanted. Upon removal from the tray cell, a significant portion of the medium does not fall out of the root system once the root system is developed. Rather, the root system and the medium form a compact structure which is removable and transplantable. The medium may be thus self-supporting, discreet medium, malleable, or conformable to various container shapes.

At the same time, the fiber should not be too dense because a dense substrate would not support an efficient germination and root development. The medium should thus be balanced with adequate water holding capacity, porosity, air space, and other properties required for optimal development in the first stages of plant propagation. In this stage of propagation, the properties may be different than when an established plant is transplanted for further growth, flowering, fruit growth, reproduction, etc.

The wood/bark fiber of the medium may have the following particle distribution described herein and shown in Table 1. In general, the wood/bark fiber may include a relatively high volume of fine fiber. The fiber may include a lower percentage of fibers of larger sizes such as fibers of sieve #8. In Table 1, the average, minimum, and maximum data is vol. % based on the total volume of the measured sample.

TABLE 1
Non-limiting example particle size distribution of
the propagation wood/bark fiber disclosed herein

| | | Propagation | Propagation | Propagation |
|---|---|---|---|---|
| | Particle | fiber - | fiber - | fiber - |
| Sieve | Range | average | minimum | maximum |
| [Mesh/μm] | [mm] | [vol. %] | [vol. %] | [vol. %] |
| #8/2360 | 2.36-4.74 | 3.5 | 2.3 | 5.0 |
| #16/1180 | 1.18-2.35 | 25.5 | 18.7 | 31.1 |
| #25/710 | 0.71-1.17 | 27.0 | 22.0 | 33.8 |
| #50/300 | 0.30-0.70 | 21.8 | 18.8 | 24.9 |
| #100/150 | 0.15-0.29 | 10.5 | 8.6 | 14.6 |
| Pan/<150 | <0.15 | 5.0 | 0.0 | 7.4 |

Non-limiting example of the propagation medium may include wood/bark fiber having about 0.20-0.50, 0.22-0.40, or 0.23-0.35, g/10 g material 8 mesh sieve. The medium may include fiber characterized by about 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50 g/10 g material 8 mesh sieve. In other words, a non-limiting example material may include about 2-5 or 2-4 vol. % of the wood/bark fiber on 8 mesh sieve such that about 2-5 or 2-4 vol. % of the wood/bark fiber remains on the #8 tray/sieve during the shaker test.

Non-limiting example of the propagation medium/material may include wood/bark fiber having about 1.9-3.11, 2.4-3.10, or 2.6-3.9 g/10 g material 16 mesh sieve. The medium may include fiber characterized by about 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 3.10, or 3.11 g/10 g material 16 mesh sieve. In other words, a non-limiting example material may include about 19-31.1 or 25-31.1 vol. % of the wood/bark fiber on 16 mesh sieve such that about 19-31.1 or 25-31.1 vol. % of the wood/bark fiber remains on the #16 tray/sieve during the shaker test.

Non-limiting example of the propagation medium/material may include wood/bark fiber having about 2.2-3.5, 2.5-3.4, or 2.6-3.3 g/10 g material 25 mesh sieve. The medium may include fiber characterized by about 2.2, 2.3, 2.4, 2.5, 2.6, 2.7, 2.8, 2.9, 3.0, 3.1, 3.2, 3.3, 3.4, or 3.5 g/10 g material 25 mesh sieve. In other words, a non-limiting example material may include about 22-33.8 or 27-34 vol. % of the wood/bark fiber on 25 mesh sieve such that about 22-33.8 or 27-34 vol. % of the wood/bark fiber remains on the #25 tray/sieve during the shaker test.

The percentage of the fiber #8 sieve is thus lower than that of the growing media designed to support plant growth in more advanced stages of plant life or the post initial propagation stages. Table 2 below provides a #8 sieve comparison of the herein-disclosed propagation fiber as Examples 1 and 2 and post initial propagation growing media, including wood, bark, or both, as Comparative Examples C1-C5.

TABLE 2
#8 sieve comparison of Examples
1 and 2 and Comparative Examples C1-C5

| | | Wood | Bark | | Typical |
|---|---|---|---|---|---|
| | | compo- | compo- | #8 | range for |
| | | nents | nents | Mesh | #8 Mesh |
| | | [wt. %] | [wt. %] | sieve | sieve |
| Example No. | | | | | |
| 1 | Propagation fiber | 100 | 0 | 0.35 | 0.3-0.4 |
| 2 | | 85 | 15 | 0.3-0.5 | 0.3-0.5 |
| Comparative | | | | | |
| Example No. | | | | | |
| C1 | Growing medium | 80 | 20 | 0.65 | 0.55-1.1 |
| C2 | for post initial | 100 | 0 | 0.65 | 0.55-1.1 |
| C3 | propagation | 80 | 20 | 1.60 | 1.3-1.9 |
| C4 | stages | 80 | 20 | 3.65 | 3.1-4.1 |
| C5 | | 100 | 0 | 3.65 | 3.1-4.1 |

In addition to the wood/bark fiber described above, the propagation medium may include the second portion, volume, weight, or mass. As was stated above, the second portion may include one or more additional types of fiber, one or more non-fibrous components, or both. The additional types of fiber and the non-fibrous components may be called supplementary materials. The first and second portions form a blend.

The supplementary materials may be included in complementary particle distribution size as the wood/bark fiber. Alternatively, one or more of the supplementary materials may have a different particle distribution than the wood/bark fiber. The amount of the supplementary materials in a blend may be about 0-90, 40-85, or 50-80 wt. or vol. %, based on the total weight or volume of the blend. The amount of the supplementary materials in a blend may be about, at least about, or at most about 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 7.5, 8, 8.5, 9, 9.5, 10, 10.5, 11, 11.5, 12, 12.5, 13, 13.5, 14, 14.5, 15, 15.5, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 wt. or vol. %, based on the total weight or volume of the blend. The blend may be free or substantially free of at least one type of the supplementary materials.

The additional types of fiber in the second portion may include fiber from one or more sources such as peat, coir, sisal, jute, straw, wheat straw, rice hulls, composted bark, alfalfa, flax, hammermilled fiber such as hammermilled tree substrate, hammermilled pine tree substrate, sawdust, compost, manure, paper, recycled paper, cellulose fibers, including newspaper, the like, or a combination thereof. The second portion may include at least one type of fiber, two types of fiber, two or more types of fiber such as 3, 4, 5, 6, 7, 8, or more types of fiber. The second portion may include more weight or volume of fiber than the first portion.

The second portion may include fiber from coconut such as coconut coir, coconut chips, coco peat, coco pith, or a combination thereof.

The second portion may include fiber of peat, sedge peat, or both. The peat may have pH of about 3-5, 3.2-4.8, or 3.5-4.5. The peat may have EC of less than about 0.25 mS/cm, assessed by the Saturated Media Extract (SME) method. The peat may have moisture content of about 40-60, 42-58, or 44-56% (w/w). The peat may have bulk density of about 4.5-10, 4.8-8, or 5-7 lb/ft³ (72.1-120.1, 76.9-115.3, or 80.1-112.1 kg/m³). The peat bulk density may be about, at least about, or at most about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0 lb/ft³.

The peat may have the following properties assessed using the North Carolina State University (NCSU) Porometer analysis, hereinafter “NCSU Porometer Analysis,” following the Procedures for Determining Physical Properties of Horticultural Substrates published by W. C. Fonteno and C. T. Harden Horticultural Substrates Laboratory Department of Horticultural Science NCSU in Raleigh, NC. The analysis implements Fisons International British Standard Procedures AFNOR Nomes (FIBSPAN) device to determine the bulk density for pack mass calculation. The test specimens are then created based on the pack mass. The analysis is used to obtain a variety of physical properties discussed herein. The peat may have total porosity of about 80-98, 85-95, or 86-92%, according to the NCSU Porometer Analysis. The peat may have porosity of about or at least about 80, 81, 82, 83, 84, 85, 86, 87, 88, 89, 90, 91, 92, 93, 94, 95, 96, 97, or 98%, according to the NCSU Porometer Analysis.

The peat may have water holding capacity (WHC) or container capacity of about 70-85, 72-82, or 75-80%. The peat may have WHC of about, at least about, or at most about 70, 71, 72, 73, 74, 75, 76, 77, 78, 79, 80, 81, 82, 83, 84, or 85%, according to the NCSU Porometer Analysis.

The peat may have air space or air pore space of about 10-20, 11-19, or 12-18%, according to the NCSU Porometer Analysis. The peat may have air space of about 10, 11, 12, 13, 14, 15, 16, 17, 18, 19, or 20%, according to the NCSU Porometer Analysis.

The peat may be screened for impurities such as sticks, resulting in a final stick quantity of about 0.7 g/2 L with max length of about 0.75″ (1.9 cm). The peat may have coarseness value of about 0.6-0.8. A non-limiting example particle distribution of the peat is listed in Table 3 below.

TABLE 3
Non-liming example particle distribution of peat

| Sieve [Mesh/μm] | Particle Range [mm] | Peat - average [vol. %] |
|---|---|---|
| #4/4750 | 4.75-6.2 | 0.5-2.0 |
| #8/2380 | 2.38-4.74 | 3.1-7.0 |
| #16/1190 | 1.19-2.37 | 10.0-17.0 |
| #25/710 | 0.71-1.19 | 16.0-22.0 |
| #50/300 | 0.30-0.70 | 25.0-32.0 |
| #100/150 | 0.15-0.29 | 16.0-22.0 |
| Pan/<150 | <0.15 | 9.0-12.0 |

Non-limiting example of the peat may include peat having about 0.3-0.7, 0.35-0.65, or 0.4-0.55 g/10 g material 8 mesh sieve. The peat may include fiber characterized by about 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, or 0.7 g/10 g material 8 mesh sieve. In other words, a non-limiting example material may include about 3-7 vol. % of the peat on 8 mesh sieve such that about 3-7 vol. % of the peat remains on the #8 tray/sieve during the shaker test.

Non-limiting example of the peat may include peat having about 1.6-2.2, 1.7-2.1, to 1.8-2.0 g/10 g material 25 mesh sieve, 100 mesh sieve, or both. The peat may include fiber characterized by about 1.6, 1.65, 1.7, 1.75, 1.8, 1.85, 1.9, 1.95, 2.0, 2.05, 2.1, 2.15, or 2.2 g/10 g material 25 mesh sieve, 100 mesh sieve, or both. In other words, a non-limiting example material may include about 16-22 vol. % of the peat on 25 mesh sieve, 100 mesh sieve, or both such that about 16-22 vol. % of the peat remains on the #25, #100, or both tray/sieve during the shaker test.

Non-limiting example of the peat may include peat having about 2.5-3.2, 2.6-3.1, or 2.7-3.0 g/10 g material 50 mesh sieve. The peat may include fiber characterized by about 2.5, 2.55, 2.6, 2.65, 2.7, 2.75, 2.8, 2.85, 2.9, 2.95, 3.0, 3.05, 3.10, 3.15, or 3.20 g/10 g material 50 mesh sieve. In other words, a non-limiting example material may include about 25-32 vol. % of the peat on 50 mesh sieve such that about 25-32 vol. % of the peat remains on the #50 tray/sieve during the shaker test.

A non-limiting example of a propagation medium having the first and second portion is shown in FIG. 6; the propagation medium includes 65 vol. % peat and 35 vol. % wood fiber. A non-limiting example of the propagation medium including wood/bark and peat fiber may have about 0.22-0.9, 0.25-0.8, or 0.3-0.7, g/10 g material 8 mesh sieve. The medium may include overall fiber characterized by about 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.9 g/10 g material 8 mesh sieve. In other words, a non-limiting example material may include about 2-9 vol. % of the wood/bark fiber on 8 mesh sieve such that about 2-9 vol. % of the wood/bark fiber remains on the #8 tray/sieve during the shaker test.

Between the first and second portion, the propagation medium may include two, three, or more types of fiber. The types may be distinguishable by the material properties, physical properties, chemical properties, or a combination thereof. For example, the medium may include fiber originating from two different natural materials. In another embodiment, the propagation medium may include fiber from just one source but be prepared differently such that one portion of the fiber has properties which are different than a second portion of the fiber.

The weight or volume ratio of the wood/bark fiber:second fiber may be about 1:99 to 99:1, 10:90 to 90:10, or 20:80 to 80:20. The weight or volume ratio of the wood/bark fiber:second fiber may be about 1:99, 2:98, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1. The weight or volume ratio of the wood/bark fiber:second:third fiber may be about 1:1:1, 1:2:1, 1:3:1, 1:2:3, 1:4:1, 2:5:7, or the like.

The supplementary materials may include heat-treated mineral particles, pigments, pigment precursors, fertilizer(s), macronutrient(s), micronutrient(s), mineral(s), mineral particle(s), biostimulant(s), interlocking manmade fiber(s), interlocking biodegradable fiber(s), surfactants, the like, or a combination thereof. The hydroponic substrate may be free of one or more of pigments, pigment precursors, fertilizer(s), macronutrient(s), micronutrient(s), mineral(s), binder(s), natural gum(s), biostimulant(s), interlocking manmade fiber(s), interlocking biodegradable fiber(s), surfactants, seed(s), the like, or a combination thereof.

The heat-treated mineral particles may include calcined particles. The calcined particles may be based on clay. The calcined clay particles may include one or more types of clay. The clay may include, for example, smectite clay(s) including the following minerals: montmorrilonite, beidellite, nantronite, saponice, hectorite. The clay may be gray, red, or both. The clay particles may be processed in the following manner for the purposes of the disclosed application. The clay may be calcined at a temperature of about 1000 to 1400, 1100 to 1350, or 1200 to 1300° F. or 537-760, 593-732, or 648-704° C. The clay may be subsequently sized or micronized, for example, by grinding. The clay may be provided in various sizes.

Fertilizers such as nitrogen fertilizers, phosphate fertilizers, potassium fertilizers, compound fertilizers, and the like may be used in a form of granules, powder, prills, or the like. For example, melamine/formaldehyde, urea/formaldehyde, urea/melamine/formaldehyde and like components may serve as a slow-release or control-release fertilizer. Fertilizers having lesser nutritional value, but providing other advantages such as improving aeration, water absorption, or being environmental-friendly may be also used. The source of such fertilizers may be, for example, animal waste, compost, and/or plant waste.

Control-release fertilizers with polyolefin, polyurethane, polymeric, bio-based, and/or biodegradable coating may be incorporated into the medium. The control-release fertilizers may be tailored for the first stage of plant propagation.

The fertilizer may be a starter fertilizer. The fertilizer may have low or substantially zero content of B, Co, Mb, or a combination thereof. The amount of the fertilizer in a blend may be about 0.25-2 lbs/yd. The fertilizer may be included in an amount of about 0-1.5, 0.1-1, or 0.2-0.8 wt. o vol. %, based on the total weight or volume of the blend. The fertilizer may be included in an amount of about, at least about, or at most about 0, 0.1, 0.2, 0.3, 0.4, 0.5, 0.6, 0.7, 0.8, 0.9, 1.0, 1.1, 1.2, 1.3, 1.4, or 1.5 wt. or vol. %, based on the total weight or volume of the blend.

Nutrients may include macronutrient, micronutrients, and minerals. Examples of macronutrients include calcium, chloride, magnesium, phosphorus, potassium, and sodium. Examples of micronutrients are also well-known and include, for example, boron, cobalt, chromium, copper, fluoride, iodine, iron, magnesium, manganese, molybdenum, selenium, zinc, vitamins, organic acids, and phytochemicals. Other macro- and micro-nutrients are well known in the art.

The mineral particle(s) may include perlite, vermiculite, sand particles, zeolite, sulfate mineral such as gypsum, hydrated aluminosilicate minerals that contain alkali and alkaline-earth metals, inorganic material including primarily calcium oxides and hydroxides such as lime, or a combination thereof. The mineral particle(s) may be treated or untreated. The lime may be dolomitic lime, hydrated lime, calcitic lime, the like, or a combination thereof. The mineral(s) may be included in an amount of about 0-5, 0.1-3.5, or 0.2-2.7 wt. o vol. %, based on the total weight or volume of the blend. The mineral(s) may be included in an amount of about, at least about, or at most about 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 wt. or vol. %, based on the total weight or volume of the blend.

Biostimulants may include any substance or microorganism that, when applied to seeds or seedling, stimulates natural processes to enhance or benefit nutrient update, nutrient use efficiency, and/or crop quality and yield. Biostimulants may include many different types. Non-limiting example biostimulants include enzymes, proteins, amino acids, protein hydrolases and/or other N-containing compounds, micronutrients such as Al, Co, Na, Se and Si, phenols, salicylic acid, monosilicic acid, humic acid, fulvic acid, seaweed extract, botanicals, biopolymers such as chitosan, inorganic compounds such as amorphous silica (SiO₂·nH₂O), silicates such as potassium silicate, calcium silicate, microbial biostimulants including mycorrhizal and non-mycorrhizal fungi, bacterial endosymbionts (like Rhizobium) and Plant Growth Promoting Rhizobacteria, fungi, etc.

Surfactants or wetting agents are compounds that decrease surface tension or interfacial tension between substances. The one or more surfactants may be nonionic, anionic, cationic, amphoteric, zwitterionic, or a combination thereof. The surfactant may assist with rewetting of the medium. The surfactant is suitable with the medium of the herein-disclosed MC. The surfactant(s) may be included in an amount of about 0-1, 0.1-0.8, or 0.2-0.0.3 wt. o vol. %, based on the total weight or volume of the blend. The surfactant(s) may be included in an amount of about, at least about, or at most about 0, 0.1, 0.2, 0.3, 0.4, 0.5, 0.6, 0.7, 0.8, 0.9, or 1.0 wt. or vol. %, based on the total weight or volume of the blend.

The propagation medium may be in a form of loose fiber, compressed fiber, a bale, slab, cube, or a different shape conformed to a container. The propagation medium may be provided in a bag, covering, loose enclosure, or a combination thereof. The propagation medium may be sold in a loose or compressed form to be added to a grow mix or a blend. The compressed form may be advantageous to increase shipping and storage efficiency. The growing medium disclosed herein may be inserted into growing containers such as trays with a plurality of cavities or cells. The growing medium may be formed into grow plugs within the trays.

A grow mix may be a propagation mix or propagation blend configured for propagation growing in one or more containers. The propagation mix may replace fully or partially traditional growing materials such as perlite. Alternatively, or in addition, the medium disclosed herein may be used for growth of more mature plants beyond the propagation stage, especially in cases where higher water holding capacity and reduction of peat volume is desired.

The propagation medium disclosed herein may be compostable, biodegradable, made from renewable resources, or a combination thereof. The herein-disclosed material presents several additional advantages such as a grower's ability to transplant at an earlier stage when compared to competitive growing materials. In other words, a lower root volume of a plant within a plug formed with the herein-disclosed material is needed before the plant may be transplanted. In at least certain embodiments, the herein-disclosed medium thus, relatively speaking, does not fall apart or crumble, does not disintegrate even if the root volume does not encompass the entire volume of the medium. In at least certain embodiments, the herein-disclosed growing medium generally retains, keeps, maintains its structural integrity throughout the propagation period.

In at least certain embodiments, the medium disclosed herein also results in rooting uniformity across a plurality of filled cells, trays, or both. In at least certain embodiments, the medium also has stable cycle of absorbing moisture with a steady dry down period, resulting in a lower amount of filled cells lost due to drying out. In at least certain embodiments, the medium is hydrophilic, not hydrophobic, and rewets faster than some traditional substrate media.

To provide desirable growing conditions for the propagation stage, in at least certain embodiments, the propagation medium disclosed herein may have the following properties. One of the characterizing properties may be loose bulk fiber density. Loose bulk fiber density may be density of the fiber laid onto the conveyor belt or into a press container prior to insertion into the grow cells. The loose bulk fiber density may be density of the laid fiber which has been manually or mechanically handled.

The loose bulk fiber density of the wood/bark fiber or the first portion may be about 1.3 to 3.0, 1.34 to 1.96, or 1.5 to 1.8 lbs/ft³ (20.8 to 48.1, 21.5 to 31.4, or 24 to 28.8 kg/m³). The loose bulk wood/bark fiber density may be about, at least about, or at most about 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.79, 1.80, 1.81, 1.82, 1.83, 1.84, 1.85, 1.86, 1.87, 1.88, 1.89, 1.90, 1.91, 1.92, 1.93, 1.94, 1.95, 1.96, 1.97, 1.98, 1.99, 2.00, 2.05, 2.1, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00 lbs/ft³.

The loose bulk fiber density of a blend may be about 5 to 12, 6 to 10, or 7 to 8 lbs/ft³ (80.1 to 192.2, 96.1 to 160.2, or 112.1 to 128.1 kg/m³). The loose bulk fiber density of a blend may be about, at least about, or at most about 5, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0 lbs/ft³.

The propagation medium may be characterized by moisture content (MC). The MC refers to the percent moisture found in a sample on a wet mass basis. MC may be calculated by: [(Wet weight-Dry weight)/Wet weight]×100. The MC denotes how much of a particular sample is comprised of water. The MC may be assessed using the NCSU Porometer Analysis, described above. Another non-limiting example way to assess MC may be via moisture scale Ohaus MB120. The loose wood/bark fiber MC may about 16-28, 19-25, or 20-22 wt. %. The loose wood/bark fiber MC may be about, at least about, or at most about 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, or 28 wt. %, according to the NCSU Porometer Analysis.

Moisture content of a blend may be about 40 to 70, 45 to 65, or 50 to 60 wt. %. Moisture content of a blend may be lower than about 60, 65, or 70 wt. %, according to the NCSU Porometer Analysis. Moisture content of a blend may be about, at least about, or at most about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 wt. %, according to the NCSU Porometer Analysis.

Total porosity of the propagation medium/wood/bark fiber/blend may be about 85-99, 88-98, or 90-97%. The total porosity of the wood/bark fiber/blend may be about or at least about 85, 86, 87, 88, 89, 90 91, 92, 93, 94, 95, 96, 97, 98, or 99%, according to the NCSU Porometer Analysis.

Container capacity or WHC of the propagation medium/wood/bark fiber/blend may be about 50-98, 60-90, or 65-85%, according to the NCSU Porometer Analysis. The container capacity of the wood/bark fiber/blend may be about 50, 52, 54, 55, 56, 58, 60, 62, 64, 65, 66, 68, 70, 72, 74, 75, 76, 77, 78, 80, 81, 82, 83, 84, 85, 86, 87, 88, 89, 90, 91, 92, 93, 94, 95, 96, 97, or 98%, according to the NCSU Porometer Analysis.

Several ratios highlight the combination of structural integrity relative to ability of the herein-disclosed growing medium to support growth.

The ratio of WHC to porosity of the propagation medium/wood/bark fiber/blend may be about 50:99% to 98:85%. The ratio of WHC to porosity of the wood/bark fiber/blend may be about, at least about, or at most about 50:99, 60:98, 65:97, 85:90, 90:88, 98:85%. The ratio of the WHC to porosity provides a substrate with optimal properties for the propagation stage of the plants disclosed herein.

The ratio of loose bulk fiber density to porosity of the wood/bark fiber is about 1.3 lbs/ft³: 99% to 3 lbs/ft³: 85%. The ratio of density to porosity of the wood/bark fiber is about, at least about, or at most about 1.3 lbs/ft³: 99%, 1.5 lbs/ft³: 97%, 1.7 lbs/ft³: 95%, 1.9 lbs/ft³: 93%, 2.2 lbs/ft³: 91%, 2.4:89%, 2.6 lbs/ft³: 87%, 2.8 lbs/ft³:86%, or 3.0 lbs/ft³: 85%.

The ratio of loose bulk fiber density to porosity of the blends is about 5 lbs/ft³: 99% to 12 lbs/ft³: 85%. The ratio of density to porosity of the wood/bark fiber is about, at least about, or at most about 5 lbs/ft³: 99%, 6 lbs/ft³: 97%, 7 lbs/ft³: 95%, 8 lbs/ft³: 93%, 9 lbs/ft³: 91%, 10:89%, 11 lbs/ft³: 87%, or 12 lbs/ft³: 85%.

The propagation medium/wood/bark fiber/blend may have volume of air space or air pore space of about 10-40, 12-38, or 14-35%, according to the NCSU Porometer Analysis. The volume of air space of the propagation medium/wood/bark fiber/blend may be about 10, 11, 12, 13, 14, 15, 16, 17, 18, 19, 20, 21, 22, 23, 24, 25, 26, 27, 28, 29, 30, 32, 34, 35, 36, 38, or 40 vol. %, according to the NCSU Porometer Analysis. The percent volume of air space may refer to air holding capacity measured as the percent volume of a material that is filled with air after the material is saturated and allowed to drain. It is the minimum amount of air the material will have.

The propagation medium/wood/bark fiber/blend may have pH of about 4.0-7.0. The propagation medium/material may have pH of about, at least about, or at most about 4.0, 4.05, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. The wood/bark fiber may have pH of about, at least about, or at most about 4.0, 4.1, 4.2, 4.3, 4.4, 4.5, 4.6, 4.7, 4.8, or 4.9. A wood/bark fiber may have pH of about 4-5, 4.1-4.9, or 4.4-4.6.

The wood/bark fiber may have electrical conductivity of less than about 0.2, 0.15, or 0.1 mS/cm, according to the SME test. The wood/bark fiber may have electrical conductivity of about or at most about 0.01, 0.02, 0.03, 0.04, 0.05, 0.055, 0.06, 0.065, 0.07, 0.075, 0.08, 0.085, 0.09, 0.095, 0.1, 0.105, 0.11, 0.115, 0.12, 0.125, 0.13, 0.135, 0.14, 0.145, 0.15, 0.155, 0.16, 0.165, 0.17, 0.175, 0.18, 0.185, 0.19, 0.195, or 0.2 mS/cm.

A blend may have electrical conductivity of less than about 0.75, 0.7, or 0.65 mS/cm, according to the SME test. A blend may have electrical conductivity of about or at most about 0.3, 0.5, 0.4, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75 mS/cm.

A method of making the disclosed propagation fiber, material, and medium is disclosed. The method may be a multi-step process. The method may include preparing initial components in the first step. For example, the initial components may be any materials named above such as fiber of the first portion, fiber of the second portion, additional materials, or a combination thereof. The fibrous materials, such as the wood fiber, may be prepared from initial materials such as chips, chunks, or large particles which are processed as described herein.

The preparing may include sizing, cleaning, sterilization, buffering, removing unwanted portions of the materials, adjusting composition of the materials, increasing or decreasing moisture content, etc.

The preparing of the wood/bark fiber may include heating of the mixture. The heating may include elevated temperatures, elevated pressure, steam, moisture, dry heat, radiator heat, microwaves, or a combination thereof. The heating may include sterilization, sanitation, elimination of microbes, spores, microorganisms, pathogens, viruses, or a combination thereof. The heating may be conducted in one or more stages, each stage having the same or different temperature and/or other conditions such as presence or absence of steam. The heating results in a softened mixture in which individual input materials may become more tender, soft, pliable, supple, or a combination thereof.

The preparing may subsequently include refining the softened mixture to the disclosed particle size using a digester. The digester may include discs which mutually cooperate to engage the softened mixture. The process may include the plates engaging, grinding, reducing in size the softened mixture into a composition having the desirable particle size disclosed herein. The refining step may include elevated temperatures, pressure, moisture, steam, or a combination thereof. The refining may include retaining the softened mixture between the discs for a predetermined amount of time such that the softened mixture is refined to the desirable particle size. The refining step may include one or more conditions which are different from the conditions of the heating step. For example, the refining step may include different pressure and/or temperature than the second step. The refining step results in the propagation fiber disclosed herein having properties disclosed herein.

The method of forming a blend includes a step of combining the first portion and the second portion to form a blend or mixture. The combining may include mixing, stirring, blending individual components. Additional method steps are anticipated such as drying, increasing moisture, adjusting one or more properties. The combining may include adding one or more supplemental components such as a surfactant, gypsum, lime, etc. to the first portion. The method may include partially compressing the propagation fiber or blend into bales, bags, towers, or containers for transportation.

A method of using the propagation medium is disclosed herein. The method may include opening/expanding the partially compressed propagation medium from bales, bags, towers, or containers for transportation. Alternatively, the method may include providing loose or uncompressed fiber/medium into bags, sacks, or other portable containers.

The method may include providing a predetermined amount of the propagation fiber/medium and filling propagation containers with the propagation fiber/medium. Example containers may include propagation trays with individual cells. The method may include filling each cell with the propagation fiber/material. The method may further include shaping the fiber/material into a 3-D medium, a plug, within the cells. The shaping may include forming a propagation fiber/material cylinder, cone, cuboid, pyramid, prism, etc. within a cell. The method may include compressing, compacting, packing the propagation fiber/medium into the cells, mechanically or automatically. The method may include providing a tray with cells for propagation of plants. The method may include forming a dibble or a small cavity in the top portion of the shaped medium within the cells for seed, cutting, seedling insertion. The dibble may be formed manually or automatically.

The process and the propagation fiber/medium may be free of any toxins and pathogens such that the process and the propagation fiber/medium comply with food safety standards, environmental standards, or both. The method may include providing seeds into the propagation medium before or after the propagation medium is inserted into the cells.

### EXAMPLES

### Examples 1, 2 and Comparative Examples C1-C5

A comparison of the properties for various growing media including the herein-disclosed propagation fiber as Examples 1 and 2 and post initial propagation growing media as Comparative Examples C1-C5 is shown in Table 4 below. Examples 1 and 2 were made by the method described above. Comparative Examples C1-C5 were produced by different methods typical for the respective products. Specific composition of Examples 1 and 2 and Comparative Examples C1-C5 is disclosed in Table 1 above.

TABLE 4
Physical properties comparison of Examples
1 and 2 and Comparative Examples C1-C5

| | Total | Container | Air | Dry Bulk |
|---|---|---|---|---|
| Example/Comp. | Porosity | Capacity | Space | Density |
| Example No. | [%] | [%] | [%] | [g/cc]/[lbs/ft³] |
| 1 | 98.71 | 79.14 | 19.57 | 0.05/2.95 |
| 2 | 99.0 | 82.00 | 17.00 | 0.05/3.50 |
| C1 | 96.56 | 69.41 | 27.15 | 0.06/3.82 |
| C2 | 93.78 | 61.84 | 31.95 | 0.06/3.71 |
| C3 | 96.56 | 56.43 | 40.13 | 0.06/3.77 |
| C4 | 95.69 | 35.00 | 60.69 | 0.06/3.52 |
| C5 | 97.02 | 32.85 | 64.17 | 0.05/3.19 |

### Examples 3-20

Examples 3-20 were prepared according to the method described above. Table 5 provides composition and particle size distribution of each Example 3-20. In Table 5, the values multiplied by 10 result in vol. % breakdown of the particles in the respective sieve sizes. Table 6 provides physical properties of Examples 3-20. Tables 5 and 6 also provide average, minimum, and maximum values for data relating to Examples 3-20.

TABLE 5
Composition and particle distribution of Examples 3-20

| | Composition | Sieves [Mesh/μm] | | | | | |
|---|---|---|---|---|---|---|---|
| Example | Wood/Bark | #8/ | #16/ | #25/ | #50/ | #100/ | |
| No. | [wt. %] | 2360 | 1180 | 710 | 300 | 150 | Pan/<150 |
| 3 | 100/0 | 0.30 | 2.52 | 2.78 | 2.34 | 1.00 | 0.63 |
| 4 | 85/15 | 0.45 | 3.04 | 2.39 | 1.96 | 0.92 | 0.34 |
| 5 | 85/15 | 0.32 | 2.69 | 2.47 | 2.27 | 1.10 | 0.68 |
| 6 | 83/17 | 0.32 | 2.87 | 2.39 | 2.12 | 1.09 | 0.67 |
| 7 | 84/16 | 0.35 | 1.97 | 3.38 | 2.37 | 1.27 | 0.07 |
| 8 | 86/14 | 0.34 | 2.77 | 2.70 | 2.12 | 0.96 | 0.52 |
| 9 | 86/14 | 0.40 | 2.60 | 3.36 | 2.03 | 0.95 | 0.00 |
| 10 | 85/15 | 0.23 | 2.33 | 2.97 | 2.43 | 1.13 | 0.60 |
| 11 | 100/0 | 0.35 | 2.75 | 2.76 | 2.09 | 0.91 | 0.43 |
| 12 | 86/14 | 0.26 | 2.45 | 2.87 | 2.25 | 0.98 | 0.56 |
| 13 | 86/14 | 0.50 | 1.99 | 2.54 | 2.31 | 1.25 | 0.74 |
| 14 | 100/0 | 0.29 | 2.61 | 2.84 | 2.17 | 0.96 | 0.56 |
| 15 | 100/0 | 0.32 | 2.38 | 2.76 | 2.26 | 1.05 | 0.56 |
| 16 | 85/15 | 0.44 | 3.11 | 2.20 | 1.88 | 0.86 | 0.43 |
| 17 | 100/0 | 0.32 | 2.45 | 2.51 | 2.13 | 1.07 | 0.56 |
| 18 | 100/0 | 0.44 | 2.82 | 2.55 | 1.97 | 0.98 | 0.49 |
| 19 | 100/0 | 0.27 | 1.87 | 2.50 | 2.49 | 1.46 | 0.70 |
| 20 | 84/16 | 0.41 | 2.74 | 2.60 | 2.05 | 0.96 | 0.50 |
| Average | | 0.35 | 2.55 | 2.70 | 2.18 | 1.05 | 0.50 |
| Minimum | | 0.23 | 1.87 | 2.20 | 1.88 | 0.86 | 0.00 |
| Maximum | | 0.50 | 3.11 | 3.38 | 2.49 | 1.46 | 0.74 |

TABLE 6
Physical properties of the Examples 3-20

| Example | Density Expanded | Moisture Content | pH | |
|---|---|---|---|---|
| No. | [lbs/ft³] | [% w/w] | [mS/cm] | EC |
| 3 | 1.54 | 17.34 | 4.1 | 0.05 |
| 4 | 1.70 | 25.55 | 4.1 | 0.06 |
| 5 | 1.80 | 26.20 | 4.1 | 0.06 |
| 6 | 1.52 | — | 4.1 | 0.07 |
| 7 | 1.52 | 19.79 | 4.1 | 0.06 |
| 8 | 1.66 | 23.85 | 4.1 | 0.07 |
| 9 | 1.34 | 16.61 | 4.1 | 0.08 |
| 10 | 1.62 | 23.37 | 4.1 | 0.07 |
| 11 | 1.36 | 17.59 | — | — |
| 12 | — | 22.16 | 4.1 | 0.07 |
| 13 | 1.74 | 22.22 | 4.0 | 0.08 |
| 14 | 1.96 | — | 4.1 | 0.07 |
| 15 | 1.58 | 20.00 | 4.0 | 0.06 |
| 16 | 1.56 | 21.15 | 4.1 | 0.07 |
| 17 | 1.52 | 21.65 | 4.1 | 0.06 |
| 18 | 1.70 | 21.29 | 4.0 | 0.08 |
| 19 | — | 18.38 | — | — |
| 20 | 1.52 | 19.51 | 4.0 | 0.06 |
| Average | 1.60 | 20.98 | 4.08 | 0.08 |
| Minimum | 1.34 | 16.61 | 4.00 | 0.05 |
| Maximum | 1.96 | 26.20 | 4.10 | 0.08 |

### Examples 21 and 22

Example blend compositions are shown below in Tables 7 and 8. The fine peat and wood/bark fiber had particle distribution disclosed above in Tables 1 and 3, respectively.

TABLE 7
Composition of Example 21

| Component name | Component volume [lbs per 1 ft³] | [wt. %] |
|---|---|---|
| Fine peat | 3.900 | 81.6 |
| Wood fiber | 0.700 | 14.6 |
| Gypsum | 0.037 | 0.8 |
| Dolomitic lime | 0.130 | 2.7 |
| Surfactant | 0.014 | 0.3 |

TABLE 8
Composition of Example 22

| Component name | Component volume [lbs per 1 ft³] | [wt. %] |
|---|---|---|
| Fine peat | 3.900 | 80.9 |
| Wood fiber | 0.700 | 14.5 |
| Gypsum | 0.037 | 0.8 |
| Dolomitic lime | 0.130 | 2.7 |
| Surfactant | 0.014 | 0.3 |
| Starter Fertilizer | 0.037 | 0.8 |

### Example 23

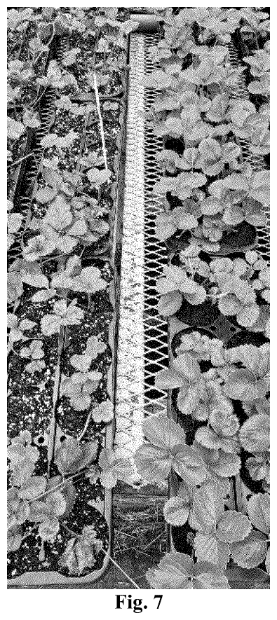

A trial using the medium disclosed herein was set up for strawberry plant propagation in trays. The Example 23 medium included a blend, composition of which included peat:wood fiber in a ratio of 65:35 vol. %. The fine peat and wood fiber had particle distribution disclosed above in Tables 1 and 2, respectively. The blend further included less than 5 wt. % of each of gypsum, lime, and surfactant. Fertilizer was not included in the blend. Example 23 was tested against a control which included peat:perlite in a ratio of 80:20. FIG. 7 is a comparative image of strawberry plants propagated in the control (on the left) and in the medium of Example 23 (on the right). The image was taken 14 days after seeding. As can be seen from FIG. 7, the strawberry plants grown in the medium of Example 23 exhibited increased growth and healthy foliage under the same watering conditions as the control. Shoot growth and rooting were superior in the medium of Example 23.

### Example 24

A trial using the medium disclosed herein was set up for begonia propagation in trays. Begonias are typically a slow crop to germinate. The Example 24 medium included a blend, composition of which included peat:wood fiber in a ratio of 70:30. The fine peat and wood fiber had particle distribution disclosed above. The blend further included less than 5 wt. % of each of gypsum, lime, fertilizer, and surfactant. The Example 24 medium was inserted into 360 cell trays.

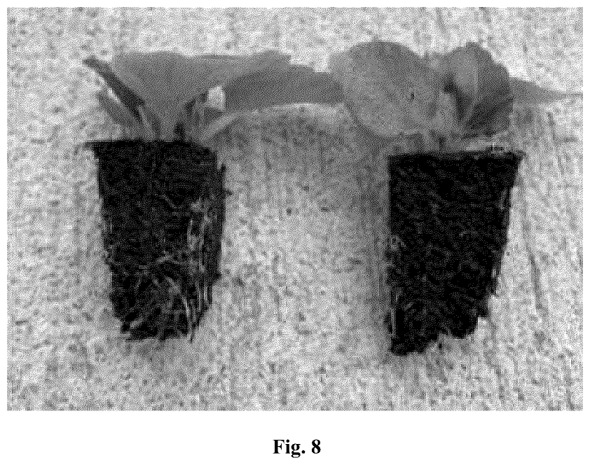

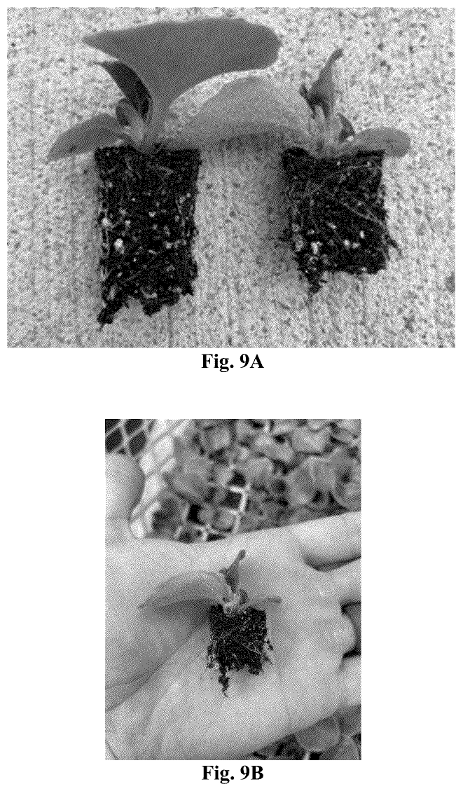

Example 24 was tested against a control which included peat:perlite in a ratio of 80:20 vol. %. The resulting growth of the begonia plants was consistently good with the plug retaining its structure after being inserted from the tray, as is shown in FIG. 8. The plugs of Example 24 retained its shape and structural integrity even when the roots did not reach the bottom of the plug. FIGS. 9A and 9B show the control, which crumbled when removed from the tray.

### Example 25

A trial using the medium disclosed herein was set up for petunia, coleus, pepper, pansy, and tomato propagation in trays. The Example 25 medium included a blend, composition of which included peat:wood fiber in a ratio of 70:30 vol. %. The fine peat and wood fiber had particle distribution disclosed above. The blend further included less than 5 wt. % of each of gypsum, lime, fertilizer, and surfactant.

The Example 25 medium was inserted into 360 (petunia, coleus, pansy, pepper, tomato) and 88 (petunia, coleus) cell trays. FIGS. 10A and 10B show example trays filled with the Example 25 medium prepared for the trials. Example 25 was tested against a control which included peat:perlite in a ratio of 80:20 vol. %.

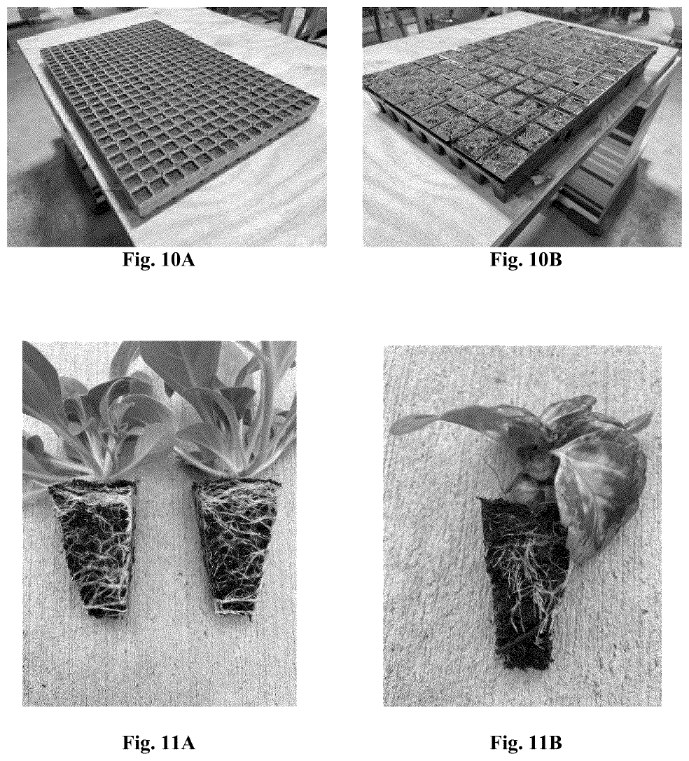

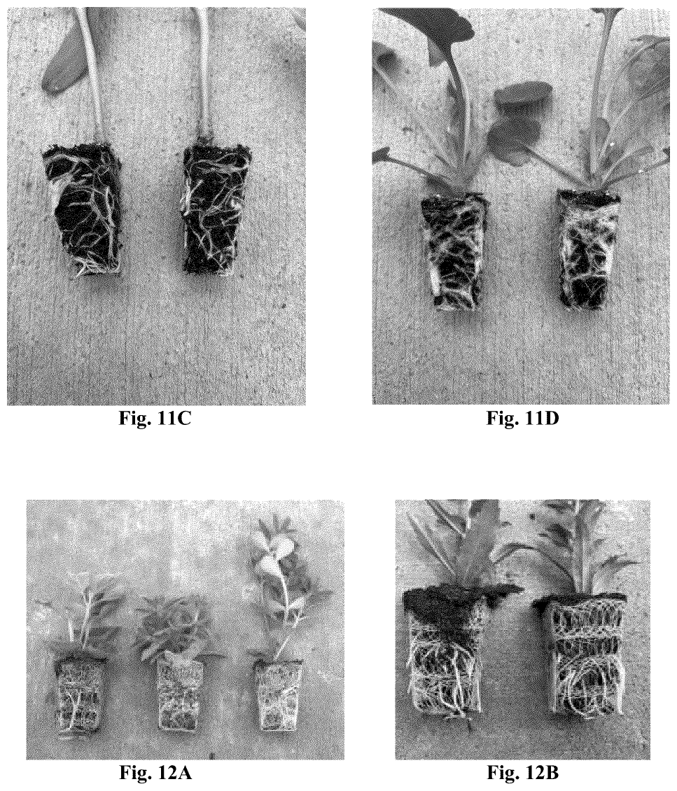

All tested species in the trays showed good propagation, good shoot growth, healthy root system. The root system of Example 25 was in some cases better than in the control. FIGS. 11A-11D demonstrate structural integrity of the plugs formed from the medium of Example 25 and good root structure of the plants grown in the medium of Example 25. FIG. 11A shows the medium with petunias. FIG. 11B shows the medium with coleus; as can be observed, the bottom tip of the plug is retained as part of the plug even after the plug was removed from the tray despite the fact that the root structure did not reach the bottom of the plug within the testing period. FIG. 11C shows the medium with peppers. FIG. 11D shows the medium with pansy plants.

### Example 26

A trial was set up to assess ability to grow plants in the medium disclosed herein with a seed plug holder. A plug holder is a structural housing typically used for propagation of various substrates. A plug holder may be woven and take shape of a cavity within a propagation tray. A plug holder may include mesh, netting, polymeric, biopolymer, or textile material.

Example 26 included trays filled with a blend disclosed herein including peat:wood/bark fiber in a ratio of 60:40 vol. % and 50:50 vol. %. FIGS. 12A and 12B show the medium of Example 26 with the seed plug holders. FIG. 12A shows Example 26 with the peat:wood/bark fiber 60:40 vol. % ratio with lantana, petunia, and portulaca (left to right). The plants developed good root system, good shoot growth, and reached the transplant stage within expected time frame. FIG. 12B shows Example 26 with the peat:wood fiber 50:50 vol. % ratio (on the left) and 60:40 vol. % ratio (on the right) with angelonia. The plants developed good root system, good shoot growth, and reached the transplant stage within expected time frame.

### Example 27

A trial using the medium disclosed herein was set up for petunia propagation in trays without any seed plug holder, netting, liner, or the like. Petunias typically develop a fine root structure, and typical growing media crumble when removed from the tray. The Example 27 medium included a blend, composition of which included peat:wood fiber in a ratio of 60:40 vol. %. The fine peat and wood fiber had particle distribution disclosed above. The blend further included less than 5 wt. % of each of gypsum, lime, fertilizer, and surfactant. Example 27 was tested against a control which included peat:perlite in a ratio of 80:20 vol. %.

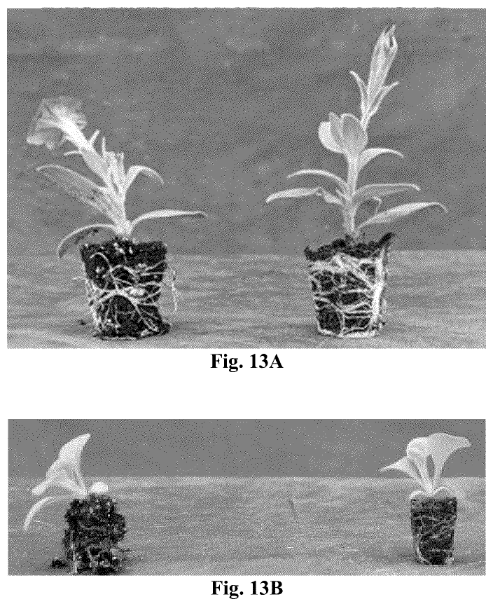

The medium of Example 27 and the control are shown in FIGS. 13A and 13B, the control being located on the left in both images. As can be observed, the control fell partially apart in the portions of the medium which was not held by the roots. In comparison, the medium of Example 27 retained its structural integrity throughout its volume.

### Example 28

A trial using the medium disclosed herein was set up for marigold and begonia propagation in trays without any seed plug holder, netting, liner, or the like. The Example 28 medium included a blend, composition of which included peat:wood fiber in a ratio of 60:40 vol. %. The fine peat and wood fiber had particle distribution disclosed above. The blend further included less than 5 wt. % of each of gypsum, lime, fertilizer, and surfactant. Example 28 was tested against a control which included peat:perlite in a ratio of 80:20 vol. %.

Erosion of substrate out of the plugs and onto floors and benches was studied. Erosion of the bottom portion of the plugs due to submersion in water and irrigation is a problematic issue for traditional growing media. The eroded sediment causes an issue with irrigation system since the deposit may accumulate and cause blockages. Additionally, the erosion may contribute to a loss of structural integrity of the growing medium and lower volume of the growing medium which the root system may develop in.

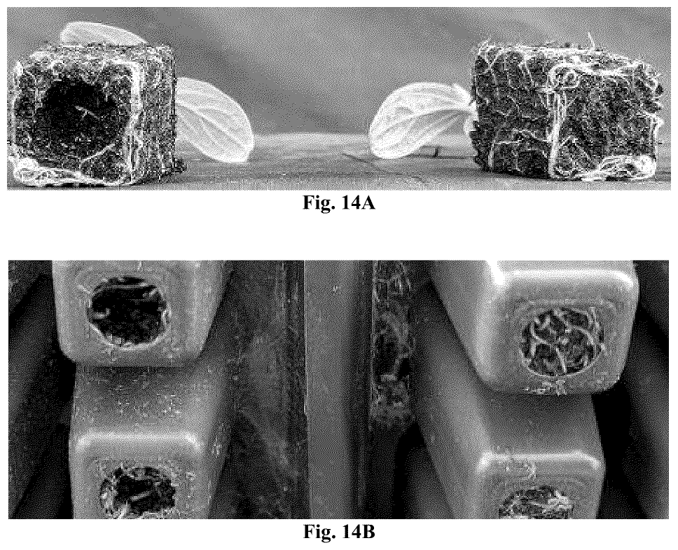

FIGS. 14A and 14B show the control on the left and Example 28 on the right for marigold growth (FIG. 14A) and begonia growth (FIG. 14B). As can be observed, the control has an eroded bottom while the bottom of Example 28 is intact, retaining its structural integrity.

### Examples 29 and 30

Two blends were prepared as Examples 29 and 30 according to the method described above. The ratio of peat:wood fiber was 65:35 vol. % for both Examples. Example 29 included 3.5 wt. % minerals and 0.3 wt. % surfactant. Example 30 also included 0.8 wt. % starter fertilizer. Table 9 lists properties of Examples 29 and 30. Table 10 provides particle distribution of the blends of Examples 29 and 30.

TABLE 9
Properties of Examples 29 and 30

| Property | Example 29 | Example 30 |
|---|---|---|
| Expanded bulk density [lbs/ft³] | 6.5 | 6.5 |
| MC [% w/w] | 49.0 | 49.0 |
| pH | 3.7 | 3.7 |
| EC [mS/cm] | <1.1 | <1.1 |
| Total porosity | 95.55 | 95.55 |
| WHC | 80.73 | 80.73 |
| Air space | 14.82 | 14.82 |
| Dry bulk density [g/cc] | 0.07 | 0.07 |
| Dry bulk density [lbs/ft³] | 4.62 | 4.62 |

TABLE 10
Particle distribution of Examples 29 and 30

| | Particle | Example 29 - | Example 30 - |
|---|---|---|---|
| Sieve | Range | average | average |
| [Mesh/μm] | [mm] | [vol. %] | [vol. %] |
| #4/4740 | 4.75-6.2 | 1.89 | 1.89 |
| #8/2360 | 2.36-4.74 | 7.02 | 7.02 |
| #16/1180 | 1.18-2.35 | 13.28 | 13.28 |
| #25/710 | 0.71-1.17 | 16.23 | 16.23 |
| #50/300 | 0.30-0.70 | 32.96 | 32.96 |
| #100/150 | 0.15-0.29 | 19.48 | 19.48 |
| Pan/<150 | <0.15 | 9.14 | 9.14 |

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the disclosure that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A fibrous propagation medium comprising:
a volume of refined, acidic wood and/or bark fiber having about 2.3-5.0 vol. % of a particle size of 2.36-4.74 mm (sieve #8), based on the total volume of the wood fiber,
moisture content of about 16-28 wt. %, and
water holding capacity of about 50-95%, according to the NCSU Promoter Analysis.

2. The fibrous propagation medium of claim 1, wherein the fibrous propagation medium further comprises about 25-31.1 vol. % of a particle size of 1.18-2.35 mm (sieve #16), based on the total volume of the wood fiber.

3. The fibrous propagation medium of claim 1, wherein the wood and/or bark fiber includes pine wood fiber.

4. The fibrous propagation medium of claim 1, wherein the fibrous propagation medium has loose bulk density of about 1.3-3.0 lbs/ft3 (20.8-48.1 kg/m3).

5. The fibrous propagation medium of claim 1, wherein the fibrous propagation medium has structural integrity when inserted into a cavity formed by a wall.

6. The fibrous propagation medium of claim 1, wherein the fibrous propagation medium is configurable into a self-supporting 3-dimensional shape without any added binder.

7. The fibrous propagation medium of claim 1, wherein the fibrous propagation medium is free of an added binder, surfactant, or both.

8. A propagation medium comprising:
a blend of
wood and/or bark fiber having about 2.3-5.0 vol. % of a particle size of 2.36-4.74 mm (sieve #8), based on the total volume of the wood fiber; and
peat having about 3.1-7.0 vol. % of a particle size of 2.36-4.74 mm (sieve #8), based on the total volume of the peat,
the propagation medium having moisture content of about 40-70 wt. % and water holding capacity of about 50-95%, according to the NCSU Promoter Analysis.

9. The propagation medium of claim 8 further comprising less than about 5 wt. or vol. % additional materials including one or more of heat-treated mineral particles, pigments, pigment precursors, fertilizer(s), macronutrient(s), micronutrient(s), mineral(s), mineral particle(s), biostimulant(s), interlocking manmade fiber(s), interlocking biodegradable fiber(s), surfactant(s), or seed(s), based on the total weight or volume of the propagation medium.

10. The propagation medium of claim 8, wherein the wood and/or bark fiber:peat vol. % ratio is about 50:50 to 90:10.

11. The propagation medium of claim 8, wherein the wood and/or bark fiber includes pine wood fiber.

12. The propagation medium of claim 8, wherein a ratio of water holding capacity to porosity of the blend is about 50:99% to 98:85%.

13. The propagation medium of claim 8, further comprising a fiber including one or more of coir, sisal, jute, straw, wheat straw, rice hulls, composted bark, alfalfa, flax, hammermilled fiber such as hammermilled tree substrate, hammermilled pine tree substrate, sawdust, compost, manure, paper, recycled paper, or cellulose fibers.

14. The propagation medium of claim 8, wherein the propagation medium has volume of air space of about 10-40%, according to the NCSU Poromoter Analysis.

15. A propagation mix comprising:
a homogenous blend of
a first volume of refined, added-binder-free wood and/or bark fiber having about 2.3-5.0 vol. % of a particle size of 2.36-4.74 mm (sieve #8), based on the total volume of the wood and/or bark fiber, moisture content of about 16-28 wt. %; and
a second volume of one or more of heat-treated mineral particles, pigments, pigment precursors, fertilizer(s), macronutrient(s), micronutrient(s), mineral(s), mineral particle(s), biostimulant(s), interlocking manmade fiber(s), interlocking biodegradable fiber(s), surfactant(s), a second type of fiber.

16. The propagation mix of claim 15, wherein the second volume includes the second type of fiber including one or more of peat, coir, sisal, jute, straw, wheat straw, rice hulls, composted bark, alfalfa, flax, hammermilled fiber such as hammermilled tree substrate, hammermilled pine tree substrate, sawdust, compost, manure, paper, recycled paper, cellulose fibers.

17. The propagation mix of claim 15, wherein the homogenous blend includes about 50-65 vol. % peat and 35-50 vol. % wood and/or bark fiber.

18. The propagation mix of claim 15, wherein the propagation mix includes seeds.

19. The propagation mix of claim 15, wherein the propagation mix has volume of air space of about 10-40%, according to the NCSU Porometer Analysis.

20. The propagation mix of claim 15, wherein the first volume:second volume ratio is about 50:50 vol. % to 90:10 vol. %.
